(19) United States
(12) Patent Application Publication
(10) Pub. No.: US 2010/0322877 A1
(43) Pub. Date: Dec. 23, 2010

(54) SILICONE ELASTOMERS IN COSMETIC ESTERS

(76) Inventors: Albert A. Zofchak, Holmdel, NJ (US); John Carson, Union City, NJ (US); Nagi M. Awad, Franklin Lakes, NJ (US); Rustico Payuno, Passaic, NJ (US)

Correspondence Address: COLEMAN SUDOL SAPONE, P.C., 714 COLORADO AVENUE, BRIDGE PORT, CT, 06605-1601, US

(21) Appl. No.: 12/734,177

(22) PCT Filed: Oct. 21, 2008
(86) PCT No.: PCT/US2008/011954

§ 371 Date: May 27, 2010

Related U.S. Application Data
(60) Provisional application No. 60/999,882, filed on Oct. 22, 2007.

(51) Int. Cl. A61K 8/37; A61Q 17/04; A61Q 15/00; A61Q 9/02; A61Q 5/12; C11D 3/37; A61Q 5/02; A61Q 19/10; A61Q 13/00

(52) U.S. Cl. 424/59; 514/552; 424/65; 424/73; 424/70.12; 512/4; 510/122; 510/158

(57) ABSTRACT

The invention describes the unexpected solubility characteristics of a number of cosmetic esters when used as solvents during the crosslinking reaction of silicone polymers to make elastomers, their use as diluting media and the uses of these ester elastomer mixtures in cosmetic and personal care products.

### RELATED APPLICATIONS

This application claims the benefit of priority of U.S. provisional patent application Ser. No. 60/999,882, of identical titled, filed Oct. 22, 2007, the entire contents of which application are incorporated by reference herein.

### FIELD OF THE INVENTION

The present invention makes use of compositions comprising crosslinked alkyl siloxane compositions and cosmetic esters as components to be used in personal care products. The present invention also relates to personal care products based upon these compositions.

### BACKGROUND

Silicone polymers are well known materials in the cosmetic and personal care industries. These materials are composed of repeating dimethylsiloxane units which may be terminated in various ways. These terminations, in large measure, determine their properties (i.e. hydrophilicity) and their ability to react with other materials. In addition to the terminal groups, the polydimethylsiloxanes themselves can have widely varying molecular weights and may be linear, branched or crosslinked in structure. Each of these variations will produce widely varying properties and uses. Some of the silicone polymers that are more useful in cosmetic and personal care products are the silicone elastomers. These are cross-linked silicone polymers that have physical properties that resemble those of rubber—in that they deform and stretch when force is applied, they bounce when dropped to the floor and they exhibit an elastic memory—exerting a force to return to their original shape when they are deformed. When diluted (or dispersed) in a solvent (or liquid) these elastomers find use as film forming materials. Further, when used in cosmetics or personal care products, the elastomer solutions or dispersions produce a very smooth, non-oily, dry feeling lubricity on skin and hair. This effect is especially appreciated in make-up products that contain pigments because the product application is greatly improved and because the elastomer film can reduce the rubbing off of the pigments once the product is applied and all of the solvents have dried. The silicone elastomers of particular interest are those described in U.S. Pat. No. 6,936,686 which teaches the preparation and use of silicone elastomers that are crosslinked as a solution in either a low viscosity silicone oil, a hydrocarbon oil (such as isododecane), a cyclomethicone or mixtures thereof. The choice of solvents is limited by the solubility/compatibility of the silicone starting polymers, the olefin, the catalyst and the final elastomer in the solvent. The same solvent that is used as a polymerization/crosslinking medium can be used to further dilute the elastomer to a desired use concentration, however, not all solvents are acceptable or compatible. For example, it is desirable for personal care product formulations to use cosmetic esters as the polymerization/crosslinking medium. However, most of the esters that are used in cosmetics are not compatible with either the reactants or the final crosslinked polymer. Nor are many of these “cosmetic esters” acceptable as diluents for the elastomers that have been crosslinked in the usual solvents (i.e. low viscosity silicone oils, hydrocarbon solvents, cyclomethicones or mixtures thereof). Surprisingly, we have found that just certain (not all) cosmetic esters can be used as a polymerization medium as well as a diluting medium. We have found that, in general, branching in the ester improves the ability of the ester to solubilize the reactants and the final crosslinked elastomer. In essence, we find that: the more branching, the better the solubilization. In addition, we have surprisingly found that the ability of a cosmetic ester to solubilize the reactants and the finished crosslinked elastomer varies with the total number of carbon atoms that it contains and reaches a maximum when the ester contains a total of eighteen carbons. The best solubilization is found when the alcohol and acid components of the ester are branched and when they have a similar number of carbons. Thus, we have found that the ester made from isononanoic acid and isononanyl alcohol provides the best solubility for both the reactants and the finished crosslinked elastomer. Experiments have also shown that the best solubilization occurs when the ester contains approximately a total of 18 carbons. Other esters may be used, but they will often cause incompatibility problems such as: increasing the time needed for the elastomer gel to form (inhibiting the crosslinking reaction), softening the resulting gel, causing the gel to ooze liquid (ester solvent), causing the gels to be cloudy or opaque, causing the reactants to be incompatible (not in complete solution) or preventing the formation of a gel entirely.

### SUMMARY AND OBJECTS OF THE INVENTION

The intention of this invention is to produce silicone based elastomer gels by the crosslinking of reactive polysiloxane materials dissolved in ester solvents that are acceptable for use in personal care products.

It is an object of this invention to crosslink siloxane polymers to form elastomeric gel products as taught in U.S. Pat. No. 6,936,686 using cosmetic esters that are commonly used in personal care products as the reaction medium and as a solvent for the finished elastomer.

It is an object of this invention to crosslink alkyl siloxane polymers to form elastomeric products as taught in U.S. Pat. No. 6,936,686 using cosmetic esters that are commonly used in personal care products as the reaction medium and as a solvent for the finished elastomer.

It is an object of this invention to produce crosslinked alkyl siloxane polymers, for example as taught in U.S. Pat. No. 6,936,686, in a limited range of esters that have a total number of carbon atoms that falls between about 12 and about 36, preferably 14 and 20. These compositions exhibit storage stability, a desirable commercial characteristic.

It is an object of this invention to produce crosslinked alkyl siloxane polymers as taught in U.S. Pat. No. 6,936,686 in a limited range of esters that have at least one component (one portion of the ester moiety either on the acyl side of the ester or on the ether (alcohol) side of the ester) that is branched rather than linear in configuration.

It is a further object of the invention to provide personal care products which make use of and include compositions comprising a crosslinked alkyl siloxane polymer in combination with a cosmetic ester.

Any one or more of these and/or other objects of the invention may be readily gleaned from a review of the description of the invention which is presented herein.

### BRIEF DESCRIPTION OF THE INVENTION

The present invention is directed to compositions comprising a crosslinked alkyl siloxane composition (“crosslinked silicone elastomer”) in combination with any cosmetic ester, more preferably about 14 to about 20 carbon atoms in size (for favorable gel formation), wherein at least one portion (i.e., the ether portion or the acyl portion of the ester) of the cosmetic ester preferably contains at least one branched-alkyl moiety in the ester. The compositions according to the present invention are preferably gels, wherein said crosslinked silicone elastomer forms a gel in the cosmetic ester. Compositions according to the present invention are preferably storage stable gels.

Compositions according to the present invention comprise an amount of a crosslinked silicone elastomer as otherwise described herein in combination or admixture with a cosmetic ester as otherwise disclosed herein, with an amount of cosmetic ester being included in admixture with said crosslinked silicone elastomer preferably effective to solubilize at least about 50% by weight of the crosslinked silicone elastomer. Preferably, the cosmetic ester solubilizes the crosslinked silicone elastomer. The admixture of crosslinked silicone elastomer and cosmetic ester may be used to produce any number of personal care products where an emollient and diluent/solvent is used. Thus, compositions according to the present invention consistent essentially of a crosslinked silicone elastomer as otherwise described herein and avoid the solvents of the prior art (i.e., a silicone oil, hydrocarbon oil or lower alkanol) in order to provide a composition with favorable attributes for including in personal care product compositions.

### DETAILED DESCRIPTION OF THE INVENTION

The following terms are used to describe the present invention. In instances where a term is not specifically defined herein, the term shall be given its meaning as understood by those of ordinary skill in the art.

The term “personal care product” is used to describe a chemical composition used for the purpose of cleansing, conditioning, grooming, beautifying, or otherwise enhancing the appearance of the human body. Personal care products include skin care products, cosmetic products, antiperspirants, deodorants, perfume, toiletries, soaps, bath oils, feminine care products, oral hygiene products, depilatories, hair-care products, including shampoos, conditioners, hair straightening products and other haircare products, color cosmetics such as lipsticks, creams, make-ups, skin creams, lotions (preferably comprised of water-in-oil or oil-in-water emulsions), shave creams and gels, after-shave lotions and shave-conditioning compositions and sunscreen products, among numerous others.

Personal care products according to the present invention comprise an admixture or polymer of a crosslinked silicone elastomer as otherwise described and a cosmetic ester comprising from about 12 to 36 carbon atoms, preferably 14 to about 20 carbon atoms and having at least one moiety (preferably two moieties) which is a branched-chain alkyl moiety within the moiety as otherwise described herein, and at least one additional component selected from the group consisting of water, an aqueous solvent (e.g. alcohol or other water compatible solvent and water), a non-aqueous solvent, emollients, humectants, oils (polar and non-polar) conditioning agents, surfactants, thickeners/thickening agents, stiffening agents, emulsifiers, medicaments, fragrances, preservatives, deodorant components, anti-perspirant compounds, skin protecting agents, pigments, dyes, coloring agents, sunscreens and mixtures thereof, among others.

Personal care products according to the present invention comprise about 0.01% to about 99.9% by weight (about 0.1% to about 95% by weight, about 0.25% to about 90% by weight, about 0.5% to about 75% by weight, about 0.5% to about 65% by weight, about 0.75% to about 50% by weight, about 1% to about 35% by weight, about 0.5% to about 25% by weight, about 0.5% to about 20% by weight, about 0.5% to about 15% by weight, about 0.25% to about 10% by weight, about 0.25% to about 7.5% by weight, about 0.1% to about 5% by weight, about 0.05% to about 2.5% by weight, etc.) of an admixture of a crosslinked silicone elastomer and a cosmetic ester as otherwise disclosed herein, the remainder of the composition comprising at least one additional component selected from the group consisting of water, an aqueous solvent (e.g. alcohol or other water compatible solvent and water), a non-aqueous solvent, emollients, humectants, oils (polar and non-polar), conditioning agents, emulsifiers, surfactants, thickeners, stiffening agents, medicaments, fragrances, preservatives, deodorant components, anti-perspirant compounds, skin protecting agents, pigments, sunscreens and mixtures thereof, among others.

In a particular aspect of the present invention, personal care compositions relate to sunscreen compositions which comprise, in addition to a crosslinked silicone elastomer and cosmetic ester, at least one additional agent selected from the group consisting octyl methoxycinnamate, octyl salicylate, menthyl anthranilate, octocrylene, benzophenone 3, avobenzone (UV absorbers), zinc oxide and titanium dioxide (pigments) and optionally, at least one additional component selected from the group consisting from the group consisting of water, an aqueous solvent (e.g. alcohol or other water compatible solvent other than a cosmetic ester and water), a non-aqueous solvent other than a cosmetic ester, emollients, humectants, oils (polar and non-polar), conditioning agents, emulsifiers, surfactants, thickeners, stiffening agents, medicaments, fragrances, preservatives, deodorant components, anti-perspirant compounds, skin protecting agents, pigments, sunscreens and mixtures thereof, among others. In sunscreen aspects of the invention, the sunscreen and/or pigment comprises about 0.01 to about 7.5% by weight, about 0.1 to about 5% by weight, about 0.5% to about 3% by weight of the final sunscreen composition, and the crosslinked silicone elastomer composition in cosmetic ester comprises about 0.1% to about 25%, about 0.25% to about 20%, about 0.5% to about 15%, about 1% to about 10% by weight of the personal care composition, wherein the crosslinked silicone elastomer composition comprises about 0.025 to about 7.5% or more, about 0.25% to about 5%, about 0.5% to about 3% by weight of said sunscreen composition.

The term “crosslinked silicone elastomer” describes a polyorganosiloxane polymer, which is generally crosslinked to produce a material that resembles rubber (resumes its original shape when a deforming force is removed). The elastomer according to the present invention may vary significantly in chemical composition but preferably is a polymeric crosslinked silicone-containing composition comprised of

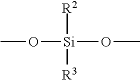

units, where R² and R³ are independently H, OH, C₁-C₁₀ (preferably C₁-C₃) alkyl or alkene groups and may vary in average molecular weight Mₙ from about 1,000 to about 1,500,000 or more, preferably about 1,000 to about 100,000, more preferably about 2,500 to about 25,000 or more, depending upon the final viscosity and other characteristics desired. Preferably R² and R³ are unreactive after final polymer formation and are preferably C₁-C₃ alkyl groups.

These final silicone elastomers are formed by reacting a first silicone elastomer pre-polymer containing two terminal vinylogous functional groups at least one functional group (preferably numerous functional groups) which preferably contain vinylogous functional groups on the terminal ends of the polymeric compound and which preferably are capable of reacting with a second silicone elastomer pre-polymer which also contains at least one and preferably numerous (approximately 5-9) functional silylhydride groups. Optionally, a third reactant, for example, a crosslinking agent or alternatively, an α,ω-diethylene terminated polydiphenyldimethylorganosiloxane is additionally added to the reaction mixture to produce final crosslinked silicone elastomer product. In preferred aspects of the invention, the first silicone elastomer pre-polymer contains (comprises) terminal vinyl groups (optionally substituted vinyl groups, acrylate or (meth)acrylate groups) and optionally, one or more Si—H groups and the second silicone elastomer pre-polymer contains (comprises) one or more Si—H groups, preferably numerous (approximately 5-9) Si—H groups (pendant from the) the polymer backbone. The crosslinking agent which may be optionally included is preferably a silicone-containing crosslinking agent may be added to the reaction mixture to produce a preferred final crosslinked silicone composition.

In addition, a mono-olefin, preferably a mono-α-olefin, such as a mono-polyalkoxylated mono-α-olefin may be added to the reaction mixture to produce grafts in the final crosslinked silicone elastomer polymer. Further details of this graft procedure may be found in U.S. Pat. Nos. 6,936,686 and 6,331,604, both of which patents are incorporated by reference herein. In general, a catalyst, preferably a platinum catalyst such as tetramethyldivinyl disiloxane platinum complex is added to the reactants. The amount of catalyst is an effective amount and the amount of catalyst may vary from as little as 0.0001% to more than 1-2% by weight of the reaction mixture (i.e., reactants plus cosmetic ester solvent), but preferably the catalyst comprises about 0.001% and 0.10% by weight or more of the final reaction mixture.

Silicone elastomer pre-polymers (i.e., polymers which have functional groups capable of crosslinking to produce crosslinked silicone elastomers according to the present invention preferably contain at least one group which is capable of reacting with a functional group on another second silicone pre-polymer such as H, OH, NH₂, or an olefinic or other alkene group. For example, with silicone elastomers which contain an Si—H group, reaction with a free hydroxyl group on a polyurethane composition produces a crosslinked Si—O-polyurethane polymer group. In the case of a Si—OH group or Si—NH₂ group, reaction with an electrophilic group on a polyurethane compound such as an isocyanate, ester group (or activated ester) or other electrophilic group will result in a crosslinked silicone elastomer-polyurethane composition. In the case of a Si-alkene group, reaction with an olefinic group or other activated group on the polyurethane can produce a silicone elastomer-polyurethane polymer crosslinked through the double bonds on each of the elastomer and polyurethane. Alternatively, the silicone elastomer and polyurethane may simply be admixed without further crosslinking/polymerization.

In preferred aspects of the present invention, the silicone elastomer comprises about 0.25% to about 99.75% by weight of the admixture of elastomer (which may be crosslinked or uncrosslinked) and cosmetic ester, preferably about 1.0% to about 99% by weight of the admixture, about 1.5% to about 98.5% by weight of the admixture, about 5% to about 95% by weight, about 10% to about 90% by weight, about 20% to about 80% by weight, about 25% to about 75% by weight, about 30% to about 70% by weight, about 35% to about 65% by weight, about 40% to about 60% by weight, about 45% to about 55% by weight, about 50% to about 55% by weight, about 3% to about 15% by weight, about 5% to about 14% by weight, about 6% to about 14% by weight, about 8% to about 14% by weight, about 8% to about 13% by weight, about 9% to about 13% by weight, about 10% to about 12% by weight, about 11% to about 12% by weight.

In certain storage stable aspects of the invention, the crosslinked silicone elastomer polymer comprises about 3% to about 15% by weight in a composition comprising silicone elastomer polymer and cosmetic ester, about 5% to about 14% by weight, about 8% to about 14% by weight, about 9% to about 13% by weight, about 10% to about 12% by weight, about 11% to about 12% by weight and wherein said cosmetic ester is between 14 and 20 carbon atoms in size, preferably 16 to 20 carbon atoms in size, 16 to 19 carbon atoms in size, 17 to 19 carbon atoms in size, 18-20 carbon atoms in size, 17-18 carbon atoms in size and 18-19 carbon atoms in size. Preferred ester compounds for use in the storage stable aspects of the invention include, for example, isononyl isononanoate (C₁₈ ester) and isooctyl isononanoate (C₁₇ ester). Compositions according to this aspect of the invention are clear (transparent) and exhibit storage stability for a period of at least about 3 months, 6 months, 9 months, 1 year, 18 months, 2 years and up to 3 years or more, at temperatures up to 50° C. (122° F.).

In certain preferred embodiments of the present invention, silicone elastomer pre-polymers have the following structure:

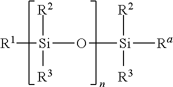

Where R¹ and Ra are independently H, an optionally substituted C₁-C₆ alkyl group (substitution with OH or a C₁-C₃ alkyl group which itself may be optionally substituted with a hydroxyl group) or an optionally substituted (preferably with C₁-C₃ alkyl or hydroxyl, preferably C₁-C₃ alkyl groups) C₂-C₆ alkenyl group (which term may include an acrylate or methacrylate group), preferably R¹ and R^{a} are optionally substituted alkenyl groups;

Each R² and R³ is independently H, OH, or a C₁-C₃ alkyl group; and

n is from 5 to 50,000, about 10 to about 25,000, about 50 to about 10,000, about 100 to about 5,000; about 150 to about 4,500, about 200 to about 4,000; about 250 to about 3500, about 250 about 2500, about 50 to about 1,000, about 75 to about 1500, about 10 to about 100, about 25 to about 750, about 25 to about 500.

Additional preferred silicone elastomer pre-polymers (second pre-polymer) for use in the present invention have the following structure:

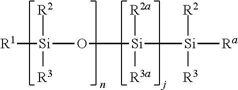

Where R¹ and R^{a} are independently H, an optionally substituted C₁-C₆ alkyl group (substitution with OH or a C₁-C₃ alkyl group which itself may be optionally substituted with a hydroxyl group) or an optionally substituted (when substituted, preferably with one ore more C₁-C₃ alkyl groups) C₂-C₆ alkenyl group (which term may include a vinyl, acrylate or methacrylate group), preferably R¹ and R^{a} are C₁-C₃ alkyl groups;

Each R² and R³ is independently H, OH, or a C₁-C₃ alkyl group (preferably a C₁-C₃ alkyl group, preferably a methyl group), preferably R² and R³ are both C₁-C₃ alkyl groups, preferably both are the same C₁-C₃ alkyl group, preferably methyl groups;

Each R^{2a} and R^{3a} is independently H, OH, or a C₁-C₃ alkyl group, preferably at least one of R^{2a} or R^{3a} is H and the other is a C₁-C₃ alkyl group;

n is from 5 to 50,000, about 10 to about 25,000, about 100 to about 10,000, about 100 to 5,000; about 500 to 5,000; about 500 to about 2,500; about 100 to about 1,000, about 150 to about 1,000; and

j is from 0 to 500, preferably 1 to about 100, about 1 to about 25, about 1 to 4, about 2 to 15 about 3 to 10; about 5 to 10.

Other preferred silicone elastomer pre-polymers for use in the present invention include polyorganosiloxane polymers which may be crosslinked with a polysiloxane (end-capped with reactive Si—H groups which react with double bonds on the uncrosslinked polysiloxane pre-polymer) crosslinking agent. Silicone elastomer pre-polymers generally range in size from an average molecular weight of at least about 500 to upwards of several million or more, about 1000 to about 1,000,000, more preferably about 1,000 to about 500,000, about 1000 to about 100,000 about 1000 to about 50,000, about 2500 to about 25,000, about 1,000 to about 5,000 about 500 to about 10,000. Final silicone elastomers in cosmetic ester solutions/admixture are generally formed by reacting a polysiloxane pre-polymer which contains at least one side chain comprising a hydrocarbon which contains a Si—H bond with another pre-polymer which contains at least one reactive double bond (e.g. a vinyl group, methacrylate, acrylate or an allyl group) which is reactive with an Si—H bond. In the present invention this reaction occurs in a cosmetic ester solution. The silicone pre-polymer may also be a crosslinker which may vary in size, but generally a crosslinker, when used, ranges in size from a molecular weight of about 125 to several thousand or more, with a preferred molecular weight range of about 200 to about 750.

The final polymer comprises crosslinked silicone elastomer polymer in combination with a cosmetic ester. The resulting gels (imbibed liquid gels), depending upon the molecular weight of the silicone elastomer contained therein, exhibit viscosities frequently in excess of 1,000,000 cps, frequently in excess of 2,000,000 cps. After application of limited shearing, the resultant gels have viscosities less than 2,000,000 cps, in the range of up to about 1,500,000 cps, in the range of up to about 1,000,000 cps, in the range up to about 750,000 cps and usually have a viscosity in excess of about 100,000 cps (although lower viscosities in particular instances are possible and within the scope of the invention depending upon the amount of cosmetic ester included in said composition), preferably in excess of about 150,000 cps. Specific viscosities can be obtained by regulation of the amount of diluent used as well as the degree of shear applied in the limited and/or controlled shearing steps. Particular non-limiting exemplary viscosity ranges within the scope of the present invention include lower limits of about 50,000 cps, about 75,000 cps, about 100,000 cps, about 150,000 cps, about 200,000 cps, about 300,000 cps, about 400,000 cps, etc and upper limits of 2,000,000 cps, about 1,500,000 cps, about 1,250,000 cps, about 1,000,000 cps, about 900,000 cps, about 800,000 cps, about 750,000 cps, etc.

Compositions according to the present invention are characterized by their clarity such that when added to other components to produce a personal care composition the resulting composition is also preferably clear. In certain preferred aspects of the invention compositions which comprise a crosslinked silicone elastomer polymer as otherwise disclosed herein

Preferred final silicone elastomers according to the present invention include for example, the silicone elastomers which are disclosed and produced in U.S. Pat. No. 6,936,686, which is incorporated by reference in its entirety herein.

The cosmetic ester and the silicone pre-polymer are mixed/admixed together to produce a final crosslinked silicone elastomer/cosmetic ester admixture or solution. In the present invention, preferably a silicone pre-polymer containing at least one functional reactive group (e.g., Si—H, Si—OH, Si—CH₂—OH, NH₂, Si—(CH₂)ⱼ—CH═CH₂ group where j is 0, 1, 2 or 3) is admixed with another silicone pre-polymer also containing at least one reactive functional group (OH, NH₂, olefin/vinyl, acrylate or methacrylate group) and then reacted to provide a crosslinked silicone elastomer/cosmetic ester composition, having varied viscosity, including viscosities consistent with gel formation. It is noted that a single silicone pre-polymer compound may be used in the present invention, the pre-polymer containing at least two crosslinkable functional groups so that the composition may be self-crosslinking. Preferably, however, at least two different silicone pre-polymers are used, one of which is silicone based and one of which may be a crosslinking agent. The silicone pre-polymers are reacted in the cosmetic ester according to the nature of the reactive functional group on the silicone pre-polymer(s) which produces a crosslinked silicone elastomer product. The pre-polymers are preferably solubilized by the cosmetic ester to produce a clear solution. The crosslinking reactions (which reactions may also include other polymerization reactions) may be conducted at temperatures at (ambient) or below room temperature, but preferably the reactions occur at elevated temperatures alone or in the presence of a catalyst and/or an acid scavenger (such as triethyl amine, etc.). The resulting crosslinked silicone elastomer polymer (especially the final viscosity) may vary according to the reaction conditions chosen to effect the crosslinking reaction and the amount of cosmetic ester used to solubilize the pre-polymers. Preferably, the final crosslinked polymer forms a gel in the cosmetic ester. Preferably, the gelled product may be stored for significant periods of times (preferably at least several weeks, more preferably at least a month, more preferably at least several months, even more preferably at least a year or more) without significantly oozing cosmetic ester from the gel composition.

The crosslinked silicone elastomer/cosmetic ester composition which is prepared above may be added to a number of components to product favorable characteristics in personal care products, including skin care products, cosmetic products, antiperspirants, deodorants, perfume, toiletries, soaps, bath oils, feminine care products, hair-care products, oral hygiene products, depilatories, including shampoos, conditioners, hair straightening products and other haircare products, color cosmetics such as lipstick, creams, make-up, skin creams, lotions and sunscreen products, among numerous others.

Compositions of the present invention may be incorporated into final personal care products as emollients for the skin and epithelial tissue such as hair, ungual tissue (nails), skin and related mucous membranes, especially given the combined attributes of emolliency and water-repellent characteristics of the crosslinked silicone elastomer. By addition of an effective amount of the present compositions, formulations for use as personal care products, including cosmetic and toiletry products will acquire a soothing and favorable interaction which promotes skin moisturization, water repellency and favorable viscosity attributes of the final personal care formulation. In addition, because the size of the crosslinked silicone elastomer and the amount of cosmetic ester can be varied substantially, numerous personal care products ranging from lotions and creams to thickened formulations to be used in stick deodorants and related products can be readily formulated.

The term “cosmetic ester” is used to describe any ester which is cosmetically compatible, i.e., may be safely incorporated into cosmetic products. In preferred aspects of the invention, a cosmetic ester has between 12 and 26 carbon atoms, about 14 and 20 carbon atoms within the ester compound, even more preferably about 16 and 19 carbon atoms within the ester compound, with a preferred cosmetic ester having at least one of the two chains, i.e., either the ether portion of the ester or the acyl portion of the ester being an optionally substituted (with alkyl or hydroxyl), preferably an unsubstituted branched-chain alkyl group. Preferred alkyl chains which correspond to the ether portion of the cosmetic ester include, for example, C₃-C₁₈ branched-chain alkyl groups, such as, for example, isopropyl, isobutyl, tert-butyl, isopentyl, neo-pentyl, branched-chain hexyl, branched-chain heptyl, branched-chain octyl, branched-chain nonyl, branched-chain decyl, branched-chain undecyl, branched-chain dodecyl, branched-chain tridecyl, branched-chain tetradecyl, branched-chain pentadecyl, branched-chain hexadecyl, branched-chain heptadecyl and branched-chain octadecyl groups linked to varying acyl groups ranging in size from C₂-C₁₇ acyl groups, accordingly. It is noted here that alternatively, the acyl group may be a branched-chain acyl group and the ether group may be an unbranched, straight chain, alkyl group. Both ether and acyl groups may comprise branched-chain alkyl groups as well.

Exemplary cosmetic esters for use in the present invention include ethyl acetate, ethyl lactate, isopropylstearate, isopropylpalmitate, isopropylmyristate, isopropyllaurate, isopropyloleate, isopropylisostearate, isononylisononanoate, isononylisoheptanoate, isononylisooctanoate, isododecylisononanoate, isooctyldodecylisononanoate, tridecylisononanoate, decylisononanoate, 2-ethylhexyl-2-ethylhexanoate, 2-ethylhexlisononanoate, isononyl-2-ethylhexanoate, isododecyl-2-ethylhexanoate, isodecyl-2-ethylhexanoate, decyl-2-ethylhexanoate, 2-ethylhexyl palmitate, 2-ethylhexyl myristate, 2-ethylhexyl laurate, 2-ethylhexyl decanoate, 2-ethylhexyl-2-butyloctanoate, 2-butyloctanyl-2-ethylhexanoate, caprylisopentanoate, laurylisopentanoate, myristylisopentanoate, palmitylisopentanoate, stearylisopentanoate, isododecylisononanoate, isooctyldodecylisononanoate isododecylneopentanoate, isooctyldodecylneopentanoate, butylmyristate, myristylbutanoate, isostearylisostearate, isostearylisononanoate, isostearylisopentanoate, isostearylisoheptanoate, diisopropyladipate, dioctyladipate, diisopropylsebacate, dioctylsebacate, isoheptyldecanoate, isoheptylisononanoate, isoheptylisopentanoate, isoheptyl-2-ethylhexanoate, dicaprylmaleate, di-2-ethylhexylmaleate, dicaprylfumerate, di-2-ethylhexylfumerate, diheptylmaleate, diisononylmaleate, diheptylfumerate and diisononyllfumerate, among numerous others.

The term “effective amount” is used throughout the present specification to describe concentrations or amounts of compounds according to the present invention which, when used in context, are effective in conveying desired traits such as emolliency, wettability, skin adherence, storage stability, and/or solubility to a formulation of a personal care product or are used to produce a compound or composition according to the present invention. In the case of the inclusion of a cosmetic ester into compositions comprising a crosslinked silicone elastomer as otherwise disclosed herein and a cosmetic ester, the amount of cosmetic ester included in compositions comprising a crosslinked silicone elastomer is effective to place the crosslinked silicone elastomer in admixture so that the composition may be readily transferred and added to additional components to produce final personal care products. The amount of cosmetic ester is preferably an amount which is effective to solubilize at least a portion of the crosslinked silicone elastomer, and preferably at least about 50% by weight of the crosslinked silicone elastomer and more preferably substantially all of the crosslinked silicone elastomer (i.e., about 85% by weight or greater), in many instances, the crosslinked silicone elastomer will be completely solubilized in the cosmetic ester and exhibit no syneresis (oozing) for a storage stable period.

The term “storage stable” is used to describe gel compositions according to the present invention which remain in a gel state and do not appreciably “ooze” cosmetic ester (exhibit syneresis) even after a period of approximately (2-3) months, even longer (6 months, 9 months, 12 months, 18 months, 2 years, 3 years or more) as otherwise described herein in storage at a temperature up to about 50° C. (122° F.).

Preferred silicone elastomers according to the present invention include for example, the silicone elastomers which are disclosed in U.S. Pat. No. 6,936,686, which is incorporated by reference in its entirety herein.

In preferred aspects, the crosslinked silicone elastomer comprises the reaction product of:
a. A first silicone elastomer pre-polymer having the following structure:

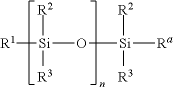

Where R¹ and Ra are independently H, an optionally substituted C₁-C₆ alkyl group or an optionally substituted C₂-C₆ alkenyl group, preferably R¹ and R^{a} are optionally substituted alkenyl groups;

Each R² and R³ is independently H, OH, or a C₁-C₃ alkyl group; and

n is from 5 to 50,000, about 10 to about 25,000, about 50 to about 10,000, about 100 to about 5,000; about 150 to about 4,500, about 200 to about 4,000; about 250 to about 3500, about 250 about 2500, about 50 to about 1,000, about 75 to about 1500, about 10 to about 100, about 25 to about 750, about 25 to about 500; and optionally an α,ω-diethylene terminated polydiphenyldimethylorganosiloxane; and
b. A second silicone elastomer pre-polymer having the following structure:

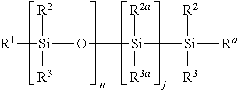

Where R¹ and R^{a} are independently H, an optionally substituted C₁-C₆ alkyl group or an optionally substituted C₂-C₆ alkenyl group, preferably R′ and R^{a} are C₁-C₃ alkyl groups;

Each R² and R³ is independently H, OH, or a C₁-C₃ alkyl group, preferably R² and R³ are both C₁-C₃ alkyl groups, preferably both are the same C₁-C₃ alkyl group, preferably methyl groups;

Each R^{2a} and R^{3a} is independently H, OH, or a C₁-C₃ alkyl group, preferably at least one of R^{2a} or R^{3a} is H and the other is a C₁-C₃ alkyl group;

n is from 5 to 50,000, about 10 to about 25,000, about 100 to about 10,000, about 100 to 5,000; about 500 to 5,000; about 500 to about 2,500; about 100 to about 1,000, about 150 to about 1,000; and

j is from 0 to 500, preferably 1 to about 100, about 1 to about 25, about 1 to 4, about 2 to 15 about 3 to 10; about 5 to 10;

and optionally, a mono-olefin to produce grafts in the final crosslinked silicone elastomer polymer, wherein said reaction product is produced in said cosmetic ester in the presence of a catalyst, preferably a platinum catalyst such as tetramethyldivinyl disiloxane platinum complex.

The present invention is now described by way of the following examples, which are illustrative of the present invention but are not to be taken as limiting the present invention in any way.

### EXAMPLES

The following examples are intended to be illustrative of the invention concepts, and are meant to provide formulas and manufacturing methods to show some of the variations and applications that are possible.

| | Example 1 | Example 2 |
|---|---|---|
| Ingredients | % w/w | % w/w |
| Dermol 99 (Isononyl Isononanoate) | 88.93 | 87.15 |
| Vinyl Terminated Polydimethyl Siloxane | 9.90 | 11.48 |
| Polydimethyl Hydrogen Siloxane | 1.10 | 1.31 |
| Alpha Olefin 12 (dodecene) | 0.01 | — |
| Catalyst (Tetramethyldivinyl disiloxane | 0.06 | 0.06 |
| Platinum complex) | 100.00 | 100.00 |

The silicone elastomers shown in these examples are made as per U.S. Pat. No. 6,936,686, (the entire contents of which is incorporated by reference herein), from a polyorganohydrosiloxane of about 3500 to 4000 molecular weight and with 6 to 7 pendant hydrosilane groups per molecule that is reacted with a 20,000 to 25,000 molecular weight vinyl terminated polydimethylsiloxane in a reaction medium of Dermol 99 at ˜11 to ˜13% w/w concentration. The reaction is catalyzed by a zero valence platinum catalyst and contains an alpha olefin as a crosslink density controller and to help make the silicone elastomer more compatible with organic solvents. After polymerization, the elastomer concentration can be further reduced by the addition of Dermol 99 using high shear mixing. Example 1 shows a preferred embodiment in which the crosslinked elastomer is reacted at 11% concentration in Isononyl Isononanoate. The Isononyl Isononanoate is pretreated with Sodium Bicarbonate to reduce moisture and to reduce the free acid content (acid value) both of which will interfere with the functioning of the catalyst. All of the reactants, except the catalyst, are added to a suitable vessel equipped with mixing that is sufficient to create a vortex. Mixing is begun such that a vortex is established without causing aeration. The reaction is heated to 35-40° C. The catalyst is added, mixing is continued and gelation occurs within about 5 minutes. Gelation is defined as the point at which the viscosity of the mixture has increased to such a point that it no longer shows movement as a fluid even though it is being continually stirred. Example 2 is another preferred embodiment in which the concentration of the reactants was increased and the alpha olefin was not used. Examples 1 and 2 produce clear elastic gels within 5 minutes of adding the catalyst.

| | Example 3 | Example 4 | Example 5 |
|---|---|---|---|
| Ingredients | % w/w | % w/w | % w/w |
| Dermol 98 (Isononyl | 88.93 | — | — |
| 2-ethylhexanoate) | | | |
| Dermol 89 (2-ethylhexyl | — | 88.93 | — |
| Isononanoate) | | | |
| Dermol IPM (Isopropyl Myristate) | — | — | 88.93 |
| Vinyl Terminated Polydimethyl | 9.90 | 9.90 | 9.90 |
| Siloxane | | | |
| Polydimethyl Hydrogen Siloxane | 1.10 | 1.10 | 1.10 |
| Alpha Olefin 12 | 0.01 | 0.01 | 0.01 |
| Catalyst (Tetramethyldivinyl | 0.06 | 0.06 | 0.06 |
| disiloxane | | | |
| Platinum complex) | 100.00 | 100.00 | 100.00 |

Examples 3 and 4 did not produce gels, showing how critical it is to have the proper solubilization of the reactants and compatibility of the finished elastomer with the solubilizing ester. The esters in these examples have a total of 17 carbon atoms. The ester in Example 5 also has a total of 17 carbon atoms, but the branching is completely different and this Example produces a clear, softer gel within 5 minutes, but it does have solvent ester oozing out after several days of storage, so, while not entirely acceptable as a commercial grade solvent, Isopropyl Myristate does make a gel where Isononyl 2-ethylhexanoate and 2-ethylhexyl Isononanoate do not. Isopropyl Myristate will also produce a gel within 5 minutes using only 0.02% catalyst. It is judged to be an acceptable solvent, but not ideal.

| | Example 6 | Example 7 | Example 8 |
|---|---|---|---|
| Ingredients | % w/w | % w/w | % w/w |
| Dermol IPP (Isopropyl Palmitate) | 88.93 | — | — |
| Dermol 109 (Isodecyl Isononanoate) | — | 88.93 | — |
| Dermol 145 (Myristyl | — | — | 88.93 |
| Neopentanoate) | | | |
| Vinyl Terminated Polydimethyl | 9.90 | 9.90 | 9.90 |
| Siloxane | | | |
| Polydimethyl Hydrogen Siloxane | 1.10 | 1.10 | 1.10 |
| Alpha Olefin 12 | 0.01 | 0.01 | 0.01 |
| Catalyst (Tetramethyldivinyl | 0.06 | 0.06 | 0.06 |
| disiloxane | | | |
| Platinum complex) | 100.00 | 100.00 | 100.00 |

The ester in Example 6 has 19 total carbons, but it did not produce a gel. However, in Example 7, the ester also has 19 carbon atoms and does produce a good gel within 5 minutes, although there is some oozing of the ester. So, Isodecyl Isononanoate is judged to be similar to Isopropyl Myristate in that it is an acceptable solvent, but not an ideal one. Example 8 is also an ester with 19 total carbons, and produced a soft gel within 5 minutes, but again, there was some oozing of the ester. So, Myristyl Neopentanoate is also judged to be similar to Isopropyl Myristate in that it is an acceptable solvent, but not an ideal one.

| | Example 9 | Example 10 |
|---|---|---|
| Ingredients | % w/w | % w/w |
| Dermol 108 (Isodecyl 2-ethylhexanoate) | 88.93 | — |
| Dermol 414 (Butyl Myristate) | — | 88.93 |
| Vinyl Terminated Polydimethyl Siloxane | 9.90 | 9.90 |
| Polydimethyl Hydrogen Siloxane | 1.10 | 1.10 |
| Alpha Olefin 12 | 0.01 | 0.01 |
| Catalyst (Tetramethyldivinyl disiloxane | 0.06 | 0.06 |
| Platinum complex) | 100.00 | 100.00 |

These two final examples show that branching plays a role in the creation of an acceptable gel. The ester in Example 9 has a total of 18 carbons (see Isononyl Isononanoate in Example 1) and branching and it does produce a soft gel within 15 minutes. This ester (Isodecyl 2-ethylhexanoate) is judged to be an acceptable solvent, but not an ideal one. Example 10 uses an ester that also has 18 total carbons, but it is linear—without branching The reactants in this Example are incompatible and no gel is produced. Butyl Myristate is not acceptable as a solvent ester for this elastomer.
As can be seen, esters are useful as solvents and as reaction media for silicone elastomer gel formation, however, they can not be used without limitation. It has been found that the preferred esters have approximately 18 total carbons and at least one chemical group (the branching can be either on the “acyl” side or the “ether” side of the ester molecule, but) within the ester that is branched.

### Silicone Elastomers in Cosmetic Esters

As can be seen, esters are useful as solvents and as reaction media for silicone elastomer gel formation, however, there are preferred esters for use in the present invention. In particular, it has been found that the preferred esters have approximately 18 total carbons and at least one chemical group that is branched.

The examples described can be used in cosmetic products as film forming agents, ingredients to promote a shiny appearance, but most importantly as ingredients to improve the application feel and slip of products. Silicone elastomers have a long history of use in cosmetic products, but they are limited by the fact that they contain materials as solvents that may be undesirable in a formulation, irritating to skin, eyes or mucous membranes or incompatible with other formulation ingredients. Using a single ester as a dilution solvent and reaction medium resolves these issues. The following non-limiting examples illustrate the use of these “cosmetic ester/elastomer” products in personal care and cosmetic formulations.

| | Example 11 | Example 12 |
|---|---|---|
| Ingredients | % w/w | % w/w |
| Example 1 - Elastomer in (Isononyl | 50.00 | 20.00 |
| Isononanoate) | | |
| Dermol 99 (Isononyl Isononanoate) | 19.50 | — |
| Elefac I-205 (Octyldodecyl Isopentanoate) | 15.00 | — |
| Octisalate | 5.00 | — |
| Octinoxate | 7.50 | — |
| Oxybenzone | 3.00 | — |
| Water | — | 73.80 |
| Acrylates/C10-30 Alkyl Acrylate | — | 1.00 |
| Crosspolymer | | |
| Cetearyl Alcohol | — | 4.00 |
| Methyliosthiazolinone (and) Iodopropynyl | — | 0.20 |
| Butylcarbamate | | |
| Triethanolamine (99%) | — | 0.40 |
| | 100.00 | 100.00 |

Example 11 shows the use of the ester/elastomer combination in an anhydrous gel formula with sunscreen ingredients and Example 12 is an elegant feeling skin cream that can be used as is or as a base for incorporating other materials for example: medicaments, moisturizers, anti-aging compounds, sunscreen ingredients, botanical extracts, fragrances, tanning ingredients and colorants such as pigments and dyes.

| | Example 13 | Example 14 |
|---|---|---|
| Ingredients | % w/w | % w/w |
| Example 7 - Elastomer in (Isodecyl | 20.00 | 30.00 |
| Isononanoate) | | |
| Minno 21 (Neopentyl Glycol | 15.00 | — |
| Diethylhexanoate & Neopentyl Glycol | | |
| Diisostearate) | | |
| Isononyl Isononanoate | — | 20.00 |
| Z-Cote (Zinc Oxide) | 20.00 | — |
| Glycereth-7 Hydroxystearate | 3.00 | — |
| Bis-PPG-12 Dimethicone IPDI | 3.00 | — |
| Water | 36.90 | 37.35 |
| Disteareth-100 IPDI | 1.00 | — |
| Bis-PEG-12 Dimethicone IPDI | 1.00 | 1.00 |
| Methylisothiazolinone (and) Iodopropynyl | 0.10 | 0.20 |
| Butylcarbamate | | |
| Acrylates/C10-30 Alkyl Acrylate | — | 0.45 |
| Crosspolymer | | |
| Various Iron Oxides & Triethoxycaprylsilane | — | 1.35 |
| Fumed Silica | — | 1.35 |
| Titanium Dioxide & Triethoxycaprylsilane | — | 8.00 |
| Triethanolamine (99%) | — | 0.40 |
| | 100.00 | 100.00 |

Example 13 shows the use of the ester/elastomer in a baby diaper cream, while Example 14 shows the use of the ester/elastomer in a make up product. Example 14 can also be made using cyclomethicone in place of the additional isodecyl isononanoate to make a “drier” feeling product and the elastomer gel can also be based upon cyclomethicone instead of the ester. In addition, film forming polymers such as Polyurethane 18 can be added to increase the “wear resistance” of the product.

| | Example 15 | Example 16 |
|---|---|---|
| Ingredients | % w/w | % w/w |
| Example 9 - Elastomer in (Isodecyl | 81.00 | 28.00 |
| 2-ethylhexanoate) | | |
| Methylheptyl Isostearate | 15.00 | — |
| Calcium Sodium Borosilicate & Titanium | 0.50 | — |
| Dioxide & Tin Oxide | | |
| Mica & Titanium Dioxide & Carmine | 2.50 | |
| 2-ethylhexyl Isononanoate | — | 10.00 |
| Elefac I-205 (Octyldodecyl Isopentanoate) | — | 10.00 |
| Dimethiconol/IPDI Coploymer | — | 20.00 |
| Alumina & Iron Oxides & | — | 7.00 |
| Triethoxycaprylsilane | | |
| Alumina & Titanium dioxide & | — | 6.00 |
| Triethoxycaprylsilane | | |
| Mica & Iron Oxides | — | 2.00 |
| Polyethylene | — | 6.00 |
| Microcrystalline wax | — | 2.00 |
| Ozokerite Wax | — | 2.00 |
| C30-50 Alcohols | — | 6.00 |
| Flavor | 1.00 | 1.00 |
| | 100.00 | 100.00 |

Example 15 illustrates the use of an ester/elastomer in a Lip gloss product that provides shine and protection for the lips. Example 16 shows the same ester/elastomer in a lip stick formula.

The preceding examples show the use of ester/elastomers in anhydrous products such as lipstick, lip balms, clear gels, sunscreen products and fragrance vehicles. In addition, the ester/elastomers can be used in many other types of anhydrous products such as: antiperspirants, hair grooms, hair styling aids, waterproof and water-resistant products. Further, examples 12, 13 and 14 show that emulsions of the ester/elastomers can be made either as a finished product or a base to which other materials can be added. These emulsions can be used as creams or lotions for skin and in hair applications such as setting, coloring and conditioning. The addition of pigments and dyes allows the hydrous emulsions to be used for coloring skin and hair emulsion products can find uses as tanning products, makeups, mascaras, fragrance vehicles and hair coloring products.

1. A composition consisting essentially of a crosslinked silicone elastomer gel in combination with a cosmetic ester.

2. The composition according to claim 1 wherein said crosslinked silicone elastomer comprises the reaction product of:
a. A first silicone elastomer pre-polymer having the following structure: 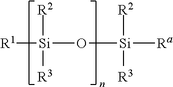
Where R¹ and Ra are independently H, an optionally substituted C₁-C₆ alkyl group or an optionally substituted C₂-C₆ alkenyl group, preferably R¹ and R^{a} are optionally substituted alkenyl groups;
Each R² and R³ is independently H, OH, or a C₁-C₃ alkyl group; and
n is from 5 to 50,000, about 10 to about 25,000, about 50 to about 10,000, about 100 to about 5,000; about 150 to about 4,500, about 200 to about 4,000; about 250 to about 3500, about 250 about 2500, about 50 to about 1,000, about 75 to about 1500, about 10 to about 100, about 25 to about 750, about 25 to about 500; and optionally an α,ω-diethylene terminated polydiphenyldimethylorganosiloxane; and
b. A second silicone elastomer pre-polymer having the following structure: 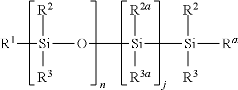
Where R¹ and R^{a} are independently H, an optionally substituted C₁-C₆ alkyl group or an optionally substituted C₂-C₆ alkenyl group, preferably R¹ and R^{a} are C₁-C₃ alkyl groups;
Each R² and R³ is independently H, OH, or a C₁-C₃ alkyl group, preferably R² and R³ are both C₁-C₃ alkyl groups, preferably both are the same C₁-C₃ alkyl group, preferably methyl groups;
Each R^{2a} and R^{3a} is independently H, OH, or a C₁-C₃ alkyl group, preferably at least one of R^{2a} or R^{3a} is H and the other is a C₁-C₃ alkyl group;
n is from 5 to 50,000, about 10 to about 25,000, about 100 to about 10,000, about 100 to 5,000; about 500 to 5,000; about 500 to about 2,500; about 100 to about 1,000, about 150 to about 1,000; and
j is from 0 to 500, preferably 1 to about 100, about 1 to about 25, about 1 to 4, about 2 to 15 about 3 to 10; about 5 to 10;
and optionally, a mono-olefin to produce grafts in the final crosslinked silicone elastomer polymer, wherein said reaction product is produced in said cosmetic ester in the presence of a catalyst.

3. The composition according to claim 1 said cosmetic ester is a C₁₄-C₂₀ cosmetic ester and said ester comprises about 85% to about 97% by weight of said composition.

4. The composition according to claim 2 wherein said reaction is conducted in solution in a cosmetic ester and said solution is clear.

5. The composition according to claim 1 wherein said cosmetic ester is selected from the group consisting of ethyl acetate, ethyl lactate, isopropylstearate, isopropylpalmitate, isopropylmyristate, isopropyllaurate, isopropyloleate, isopropylisostearate, isononylisononanoate, isononylisoheptanoate, isononylisooctanoate, isododecylisononanoate, isooctyldodecylisononanoate, tridecylisononanoate, decylisononanoate, 2-ethylhexyl-2-ethylhexanoate, 2-ethylhexlisononanoate, isononyl-2-ethylhexanoate, isododecyl-2-ethylhexanoate, isodecyl-2-ethylhexanoate, decyl-2-ethylhexanoate, 2-ethylhexyl palmitate, 2-ethylhexyl myristate, 2-ethylhexyl laurate, 2-ethylhexyl decanoate, 2-ethylhexyl-2-butyloctanoate, 2-butyloctanyl-2-ethylhexanoate, caprylisopentanoate, laurylisopentanoate, myristylisopentanoate, palmitylisopentanoate, stearylisopentanoate, isododecylisononanoate, isooctyldodecylisononanoate isododecylneopentanoate, isooctyldodecylneopentanoate, butylmyristate, myristylbutanoate, isostearylisostearate, isostearylisononanoate, isostearylisopentanoate, isostearylisoheptanoate, diisopropyladipate, dioctyladipate, diisopropylsebacate, dioctylsebacate, isoheptyldecanoate, isoheptylisononanoate, isoheptylisopentanoate, isoheptyl-2-ethylhexanoate, dicaprylmaleate, di-2-ethylhexylmaleate, dicaprylfumerate, di-2-ethylhexylfumerate, diheptylmaleate, diisononylmaleate, diheptylfumarate, diisononyllfumarate, and mixtures thereof.

6. A storage stable composition consisting essentially of an admixture of a crosslinked silicone elastomer polymer in combination with a cosmetic ester, wherein said cosmetic ester comprises about 85% to about 97% by weight of said composition and said crosslinked silicone elastomer polymer comprises about 3% to about 15% by weight of said composition.

7. The composition according to claim 6 wherein said cosmetic ester is a C₁₄-C₂₀ ester.

8. The composition according to claim 6 wherein said cosmetic ester is a C₁₆-C₂₀ ester.

9. The composition according to claim 6 wherein said cosmetic ester is a C₁₆-C₁₉ ester.

10. The composition according to claim 6 wherein said cosmetic ester is isononyl isononanoate, isooctyl isononanoate and mixtures thereof.

11. The composition according to claim 6 wherein said cosmetic ester comprises about 87% to about 91% by weight.

12. The composition according to claim 6 wherein said cosmetic ester comprises about 88% to about 89% by weight.

13. The composition according to claim 6 wherein said cosmetic ester comprises about 88% to about 90% by weight.

14. The composition according to claim 1 in the form of a gel.

15. The composition according to claim 6 wherein said crosslinked silicone elastomer comprises the reaction product of:
a. A first silicone elastomer pre-polymer having the following structure: 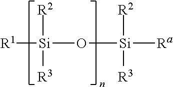
Where R¹ and R^{a} are independently H, an optionally substituted C₁-C₆ alkyl group or an optionally substituted C₂-C₆ alkenyl group, preferably R¹ and R^{a} are optionally substituted alkenyl groups;
Each R² and R³ is independently H, OH, or a C₁-C₃ alkyl group; and
n is from 5 to 50,000, about 10 to about 25,000, about 50 to about 10,000, about 100 to about 5,000; about 150 to about 4,500, about 200 to about 4,000; about 250 to about 3500, about 250 about 2500, about 50 to about 1,000, about 75 to about 1500, about 10 to about 100, about 25 to about 750, about 25 to about 500; and optionally an α,ω-diethylene terminated polydiphenyldimethylorganosiloxane; and
b. A second silicone elastomer pre-polymer having the following structure: 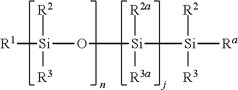
Where R¹ and R^{a} are independently H, an optionally substituted C₁-C₆ alkyl group or an optionally substituted C₂-C₆ alkenyl group, preferably R¹ and R^{a} are C₁-C₃ alkyl groups;
Each R² and R³ is independently H, OH, or a C₁-C₃ alkyl group, preferably R² and R³ are both C₁-C₃ alkyl groups, preferably both are the same C₁-C₃ alkyl group, preferably methyl groups;
Each R^{2a} and R^{3a} is independently H, OH, or a C₁-C₃ alkyl group, preferably at least one of R^{2a} or R^{3a} is H and the other is a C₁-C₃ alkyl group;
n is from 5 to 50,000, about 10 to about 25,000, about 100 to about 10,000, about 100 to 5,000; about 500 to 5,000; about 500 to about 2,500; about 100 to about 1,000, about 150 to about 1,000; and
j is from 0 to 500, preferably 1 to about 100, about 1 to about 25, about 1 to 4, about 2 to 15 about 3 to 10; about 5 to 10;
and optionally, a mono-olefin to produce grafts in the final crosslinked silicone elastomer polymer, wherein said reaction product is produced in said cosmetic ester in the presence of a catalyst.

16. The composition according to claim 1 wherein at least one of the moieties of the cosmetic ester contains a branched-chain alkyl group.

17. The composition according to claim 1 wherein said cosmetic esters produce clear solutions of the reactants and product.

18. The composition according to claim 2 wherein said catalyst comprises between about 0.001% and 0.10% by weight.

19. A personal care composition comprising a composition according to claim 1 in combination with at least one additional component selected from the group consisting of water, an aqueous solvent, a non-aqueous solvent, emollient, humectant, oil, conditioning agent, surfactant, thickener/thickening agent, stiffening agent, emulsifier, medicament, fragrance, preservatives, deodorant component, anti-perspirant compound, skin protecting agent, pigment, dye, coloring agent, sunscreen and mixtures thereof.

20. The composition according to claim 19 which is a skin care product, color cosmetic product, antiperspirant, deodorant, perfume, toiletry, soap, bath oil, feminine care product, hair-care product, oral hygiene product, depilatory, shampoo, conditioner, hair straightening product, lipstick, skin cream, make-up, lotion, shave cream, shave gel, after-shave lotion, shave-conditioning composition and sunscreen product.

21. A sunscreen composition comprising a composition according to claim 1 in an amount ranging from about 0.1% to about 25% by weight in combination with at least one sunscreen agent or pigment selected from the group consisting octyl methoxycinnamate, octyl salicylate, menthyl anthranilate, octocrylene, benzophenone 3, avobenzone, zinc oxide, titanium dioxide and mixtures there of in an amount ranging from about 0.01% to about 7.5% by weight, and optionally, at least one additional component selected from the group consisting from the group consisting of water, an aqueous solvent, a non-aqueous solvent other than a cosmetic ester, emollients, humectants, oils, conditioning agents, emulsifiers, surfactants, thickeners, stiffening agents, medicaments, fragrances, preservatives, deodorant components, anti-perspirant compounds, skin protecting agents, pigments, sunscreens and mixtures thereof.

22. The composition according to claim 21 wherein said sunscreen agent or pigment comprises about 0.5% to about 3% by weight.

23. The composition according to claim 21 wherein crosslinked silicone elastomer polymer comprises about 0.025% to about 7.5% by weight.
